(19) United States
(12) Patent Application Publication
(10) Pub. No.: US 2023/0338497 A1
(43) Pub. Date: Oct. 26, 2023

(54) METHOD FOR PREPARING A VACCINE COMPOSITION FROM LYOPHILIZED ANTIGENS

(71) Applicant: VAXINANO, Loos (FR)

(72) Inventor: Didier Betbeder, Houplin Ancoisne (FR)

(21) Appl. No.: 18/004,980

(22) PCT Filed: Jul. 8, 2021
(86) PCT No.: PCT/FR2021/051270

§ 371 Date: Jan. 10, 2023

(30) Foreign Application Priority Data
Jul. 10, 2020 (FR) FR2007338

(51) Int. Cl. A61K 39/108; A61K 9/19

(52) U.S. Cl. CPC A61K 39/0258; A61K 9/19; A61K 2039/55555

(57) ABSTRACT

The field of extemporaneous preparation of vaccine compositions from lyophilized antigens. More specifically, the present disclosure relates to the use of cationic nanoparticles to render the lyophilized antigens more soluble without adding a lyophilization aid, with a view to extemporaneous use for administering a vaccine composition. In a particular embodiment, the present disclosure allows a vaccine formulation to be prepared or one or more valencies to be added to a previously formulated vaccine composition.

### CROSS-REFERENCE TO RELATED APPLICATIONS

This application is a national phase entry under 35 U.S.C. § 371 of International Patent Application PCT/FR2021/051270, filed Jul. 8, 2021, designating the United States of America and published as International Patent Publication WO 2022/008848 A1 on Jan. 13, 2022, which claims the benefit under Article 8 of the Patent Cooperation Treaty to French Patent Application Serial No. FR2007338, filed Jul. 10, 2020.

### TECHNICAL FIELD

The present disclosure relates to the field of extemporaneous preparation of a vaccine composition from lyophilized antigens. More particularly, the present disclosure relates to the use of cationic nanoparticles to solubilize the lyophilized antigens without adding a lyophilization aid, with a view to extemporaneous use for administering a vaccine composition. In a particular embodiment, the present disclosure allows a vaccine formulation to be prepared or one or more valencies to be added to a previously formulated vaccine composition.

### BACKGROUND

The prior art relating to nanoparticles, in particular, teaches their uses for increasing the infectious capacity of a non-enveloped virus, as described in the application WO2018/104762A1. From the prior art, from the application EP2708237A1, a pharmaceutical composition is also known comprising, as an active ingredient, a mixture of: (i) a solid nanoparticle comprising a cationic polysaccharide core, the core being porous and charged with at least one anionic phospholipid; and (ii) at least one antigen obtained from an intracellular pathogen; and (iii) a pharmaceutically acceptable solvent.

Molecular vaccine transport means are also known from the prior art. The application WO98/29099A2 can be cited, which discloses a method for the administration into the mucous membranes of a substance to a mammal, by virtue of a biovector which comprises a natural polymer, or a derivative or a hydrolysate of a natural polymer, or a mixture thereof. The application FR2803526A1 can also be cited, which discloses a polymer matrix which is characterized in that it comprises a macromolecular hydrophilic matrix bearing a positive or negative ionic charge, and into which a lipidic phase of a sign contrary to that of the matrix is incorporated. The matrix can, in particular, be used for transporting antigens for vaccine use.

The interactions between the nanoparticles and epithelial cells of the respiratory tracts, their ability to cross the barrier, and the analysis of the impact of lipids inside the nanoparticles in the release and transcytosis of antigens in the epithelial cells have been studied in the study by BERNOCCHI B et al., “Mechanisms allowing protein delivery in nasal mucosa using NPL nanoparticles” JOURNAL OF CONTROLLED RELEASE, vol. 232, 11 Apr. 2016. The study mentions particles formed of cross-linked cationic polysaccharides comprising an anionic phospholipid or not.

Albumin is conventionally used for solubilizing small lipophilic molecules. In this respect, the application FR3005858A1 can be cited, which describes the preparation of soluble anti-malarial compositions comprising nanoparticles. In this composition, the albumin is used as a solubilizing agent for the anti-malarial molecule, and the authors show that the therapeutic properties are not lost.

Other lyophilization aids, such as arginine and histidine, have also been described as facilitating the solubilization of lyophilized proteins. The application EP0638091A1 describes the preparation of a lyophilized complex factor VIII composition which, upon contact with water, is reconstituted in less than a minute, at ambient temperature. The preparation comprises (i) adding a solubilizing agent comprising arginine, in an amount sufficient for increasing the solubility of the factor VIII, and histidine, to the aqueous solution, in order to thus form a factor VIII/arginine/histidine solution in which the histidine is present in the factor VIII/arginine/histidine solution at a concentration of 0.025 M, and (ii) lyophilizing the factor VIII/arginine/histidine solution in order to thus provide a complex factor VIII composition having increased solubility. The application EP2458990A1 also proposes using arginine and histidine, but also saccharose and mannitol.

However, these lyophilization aids may have undesirable effects, in particular, if the extemporaneous composition reconstituted from lyophilized proteins is intended for vaccine use.

In the context of the formulation of vaccine compositions, the solubilization of lyophilized proteins poses problems on account of the heterogeneity of features of the antigens, or the presence of aids or additives associated with the proteins.

To date, there is no method available which is suitable for the solubilization of lyophilized proteins and which does not require the addition of an aid compatible with an administration as a vaccine composition.

### BRIEF SUMMARY

A method has been developed that makes it possible to easily and rapidly prepare a vaccine composition from lyophilized antigens; the method is based on the use of cationic nanoparticles solubilized in an aqueous medium. In contrast with conventional solubilization methods, the method does not require the addition of a lyophilization aid.

Thus, the present disclosure relates to a method for preparing a vaccine composition from a lyophilized antigen, comprising the steps of:
providing an aqueous solution comprising a cationic nanoparticle consisting of a cationic polysaccharide core and associated or not with antigenic proteins
solubilizing the lyophilized antigen in the aqueous solution
incubating the composition thus obtained at ambient temperature.

Advantages of the present disclosure include the following.

The solubilization method is simple and rapid; indeed, it is sufficient to re-suspend the lyophilized antigens in the solution of nanoparticles, to mix them, and to leave them to incubate at ambient temperature. An extemporaneous composition is thus obtained in less than 30 minutes.

The composition does not contain any solubilizing agent (other than the nanoparticles themselves), which prevents the undesirable effects associated with this type of molecule. When the vaccine composition contains only lyophilized antigens, it does not contain any vaccine additive either. This is advantageous since the mineral additives (i.e., mineral salts such as aluminum salts) remain in the body for a very long time (several decades).

The antigens combine with the nanoparticles in solution and are released in the region of the immune cells following administration, whereas the nanoparticles are rapidly eliminated (in less than 72 hours following nasal administration). The nanoparticles thus play a quadruple role—solubilizing agent, stabilizing agent and transport vector for antigens, and vaccine additive.

By implementing the method of the present disclosure, the solubilization of hydrophobic or hydrophilic proteins is achieved, by virtue of the nanoparticles, whatever the size of the proteins. The small proteins are absorbed by the nanoparticles. The large proteins, in turn, are surrounded by the nanoparticles, which prevents them from joining together. In the case of a vaccine composition, this makes it possible to administer antigens of different sizes, for example, a mixture of antigens obtained by crushing the whole pathogen. Thus, the antigenic presentation has a broad spectrum, and the chances of success of the vaccination are increased compared with conventional approaches based on the selection of an antigen or mixture of specified or poorly represented antigens.

By virtue of the nanoparticles, it is no longer necessary to use solubilization aids, which are not compatible with a preparation for vaccine use.

Thus, the present disclosure represents a technological advancement in the field of vaccines. Indeed, the antigen preparations in solution which are used today are not stable for more than a few months, and thus have to be produced regularly, with the unused stocks of antigens being destroyed. The present disclosure makes it possible to provide a solution to this problem by making it possible to solubilize antigens kept for months or years in lyophilized form. It is thus possible, by virtue of using cationic nanoparticles, to extemporaneously (re)solubilize any antigenic preparation kept in lyophilized form. Thus, the management of antigen preparation stocks is improved, allowing for a reduction in costs and greater flexibility in the preparation of vaccines.

The fact of being able to solubilize the lyophilized antigens makes it possible to combine different antigens or antigenic preparations in order to produce vaccines, the targets of which are adjustable: preparation of combined vaccines, but also vaccines of which the valency is adjusted depending on the geographical distribution of the pathogen strains.

Another advantage of the present disclosure is that it makes it possible to add antigens to existing vaccines in order to increase the valency thereof. In order to achieve this, the antigens to be added, available in lyophilized form, are added to the existing vaccine composition to which cationic nanoparticles have previously been added.

### BRIEF DESCRIPTION OF THE DRAWINGS

FIG. 1: PAGE in native conditions of compositions comprising bovine serum albumin (BSA) or a total extract of E. coli (ET) and cross-linked nanoparticle (NP) charged with lipids (NPLs), as well as the addition of BSA/NPL in a composition comprising ET.

FIG. 2: PAGE in native conditions of compositions comprising BSA or a total extract of E. coli (ET) and NPLs, as well as the addition of ET/NPL in a composition comprising BSA.

FIG. 3: PAGE in native conditions of compositions comprising BSA or a total extract of E. coli (ET) and non-cross-linked NP charged with lipids (NPL NRs), as well as the addition of BSA/NPL NR in a composition comprising ET.

FIG. 4: PAGE in native conditions of compositions comprising BSA or a total extract of E. coli (ET) and NPL NRs, as well as the addition of ET/NPL NR in a composition comprising BSA.

FIG. 5: PAGE in native conditions of compositions comprising BSA or a total extract of E. coli (ET) and cross-linked NP not charged with lipids (NP+), as well as the addition of BSA/NP+ in a composition comprising ET.

FIG. 6: PAGE in native conditions of compositions comprising BSA or a total extract of E. coli (ET) and NP+, as well as the addition of ET/NP+ in a composition comprising BSA.

FIG. 7: PAGE in native conditions of compositions comprising BSA or a total extract of E. coli (ET) and non-cross-linked NP not charged with lipids (NP+NRs), as well as the addition of BSA/NP+ NR in a composition comprising ET.

FIG. 8: PAGE in native conditions of compositions comprising BSA or a total extract of E. coli (ET) and NP+ NRs, as well as the addition of ET/NP+ NR in a composition comprising BSA.

### DETAILED DESCRIPTION

The present disclosure relates to a method for preparing a vaccine composition from at least one lyophilized antigen, comprising the steps of:
providing an aqueous solution comprising a cationic nanoparticle consisting of a cationic polysaccharide core, the nanoparticle being associated or not with antigenic proteins
adding the lyophilized antigen to the aqueous solution
incubating the composition thus obtained at ambient temperature.

The aqueous solution comprises the nanoparticles required for the solubilization of the lyophilized antigens. The addition of the aqueous solution, and the incubation, allow for the solubilization of the lyophilized antigen.

In one embodiment, the method according to the present disclosure makes it possible to increase the valency of a monovalent or multivalent vaccine composition. “Valency” means the part of a vaccine corresponding to the protection against a single germ. A multivalent vaccine can protect against a plurality of germs which cause the same disease (such as the 13-valent vaccine against pneumococcus) or against different diseases (such as the MMR vaccine).

In such an embodiment, the aqueous solution comprises at least one antigenic protein. In this case, the antigenic protein (of the initial vaccine composition) is associated with nanoparticles prior to addition of the lyophilized antigen. It is possible to add one or more lyophilized antigens to the initial vaccine composition.

“Cationic nanoparticle consisting of a cationic polysaccharide core” means a solid nanoparticle (NP) comprising a cationic polysaccharide core. The NP may be cross-linked or not. The core thereof may be charged or not with an anionic phospholipid. The NP is not surrounded by any phospholipidic layer.

In a first particular embodiment, the cationic polysaccharide forming the core of the NP is a non-cross-linked polymer obtained by the reaction between a polysaccharide selected from starch, dextran, dextrin and maltodextrin, poly-fructoses (inulin), poly-mannoses, poly-galactoses, poly-galactomannans (guar gum), and at least one cationic ligand selected from a primary, secondary or tertiary amine, or quaternary ammoniums. The core is not charged with lipids.

In a second particular embodiment, the cationic polysaccharide forming the core of the NP is a cross-linked polymer obtained by the reaction between a polysaccharide selected from starch, dextran, dextrin and maltodextrin, poly-fructoses (inulin), poly-mannoses, poly-galactoses, poly-galactomannans (guar gum), and at least one cationic ligand selected from a primary, secondary or tertiary amine, or quaternary ammoniums, and then addition of a cross-linking agent. The cross-linking agent is selected from epichlorohydrin, a dicarboxylic acid or an acid chloride, such as sebacic acid. The core is not charged with lipids.

In a preferred embodiment, the cationic polysaccharide is obtained by the reaction between the maltodextrin and the glycidyltrimethylammonium, whether the NP is cross-linked or not.

In a third particular embodiment, the cationic polysaccharide forming the core of the NP is charged with an anionic phospholipid. The anionic phospholipid may be selected from diacylphosphatidyl glycerol, diacylphosphatidyl serine, or diacylphosphatidyl inositol. In another preferred embodiment, the anionic phospholipid is dipalmitoylphosphatidylglycerol (DPPG). The cationic polysaccharide forming the core of the NP is not cross-linked.

In an entirely preferred embodiment, the NP is a nanoparticle of maltodextrin charged with DPPG.

In a fourth particular embodiment, the cationic polysaccharide forming the core of the NP is not charged with lipids and is not cross-linked.

The embodiments according to which the NPs are cross-linked or not, and charged with lipids or not, can be combined, in order to give four types of NP:
cross-linked NP not charged with lipids (NP+)
cross-linked NP charged with lipids (NPL)
non-cross-linked NP not charged with lipids (NP+NR)
non-cross-linked NP charged with lipids (NPL NR)

These four types of NP can be used in the method according to the present disclosure.

In a preferred embodiment, the NPs are used in solution, in order to solubilize a lyophilized antigenic formulation. Such a formulation may be ready for use or customized from different lyophilized antigens.

In a particular embodiment of the present disclosure, the method for preparing a composition according to the present disclosure makes it possible to add one or more valencies to an existing vaccine composition, already formulated. In this case, the aqueous solution in which the lyophilized antigen is resuspended is a vaccine composition comprising at least one other antigen already in solution, associated with the cationic nanoparticle. The steps of the method consist in:
providing a vaccine composition
adding NPs consisting of a cationic polysaccharide core as defined above
re-suspending at least one additional lyophilized antigen in the vaccine composition containing the NPs
incubating the composition thus obtained at ambient temperature.

In this embodiment, the present disclosure relates to a method for adding a new valency to a vaccine composition, and can be defined by the steps of:
providing an aqueous vaccine composition of a nanoparticle consisting of a cationic polysaccharide core containing associated antigens
re-suspending at least one additional lyophilized antigen in the aqueous vaccine composition
incubating the composition thus obtained at ambient temperature.

This method is particularly advantageous for preparing multivalent vaccines from already formulated vaccines, which was hitherto not possible.

Within the meaning of the present disclosure, “lyophilized antigen” means an antigenic protein, a mixture of antigenic proteins, or a partial or total pathogen extract. The pathogen extract may contain proteins, polysaccharides and lipids. The protein may be hydrophilic or lipophilic. The antigens may be purified, alone or in combination. The antigenic proteins may be lipophilic or hydrophilic.

In a preferred embodiment, the mixture of antigenic proteins is made up of one or more purified antigens or a pathogen extract. The pathogen extract may be a total extract or a partial extract.

In a preferred embodiment, the antigen is a complex extract of proteins obtained from a whole pathogen.

The pathogen may be a parasite, a virus, a bacterium, a mycobacterium, and a fungus. The pathogens of interest include the following examples:
(i) a virus selected from herpes simplex virus 1 and 2, human papillomavirus, cytomegalovirus, mycobacterium tuberculosis, dengue fever, HIV, respiratory syncytial virus (RSV), hepatitis A virus, hepatitis B virus and hepatitis C virus, a coronavirus such as SARS-Cov2, rabies virus, or a veterinary virus such as African horse sickness virus, African swine fever virus, Andes virus, avian influenza virus, equine influenza virus, bluetongue virus, Chapare virus, Chikungunya virus, Choclo virus, Crimean-Congo hemorrhagic fever virus, dengue virus, Dobrava-Belgrade virus, eastern equine encephalitis virus, Ebola virus, foot-and-mouth disease virus, capripox virus, Guanarito virus, Hantaan virus, Hendra virus (equine morbillivirus), porcine herpesvirus (Aujeszky's disease virus), equine herpesvirus, classical swine fever virus, Japanese encephalitis virus, Junin virus, Kyasanur forest disease, Laguna Negra virus, Lassa fever virus, louping ill virus, Lujo virus, lumpy skin disease virus, lymphocytic choriomeningitis virus, Machupo virus, Marburg virus, monkeypox virus, Murray Valley encephalitis virus, Newcastle disease virus, Nipah virus, Omsk hemorrhagic fever virus, Oropouche virus, ovine rinderpest virus, porcine enterovirus 9 (swine vesicular disease virus), Powassan encephalitis virus, rabies virus and other members of the Lyssavirus genus, Rift Valley fever virus, rinderpest virus, Rocio virus, Sabia virus, Seoul virus, sheep pox virus, Sin Nombre virus, Saint Louis encephalitis virus, Teschen disease virus (enterovirus encephalomyelitis), porcine reproductive and respiratory syndrome virus, tick-borne encephalitis virus (Russian Spring-Summer encephalitis virus), smallpox virus, Venezuelan equine encephalitis virus, vesicular stomatitis virus, Western equine encephalitis virus, yellow fever virus, and feline leukemia virus.
(ii) an intracellular parasite selected from Acanthamoeba spp., Babesia spp., Balantidium coli, Blastocytis, Dientamoeba fragiiis, Entamoeba histolytica, Giardia lamblia, Isospora belli, Leishmania spp., Naegleria fowleri, Rhinosporidium seeberi, Trichomonas vaginalis, Trypanosoma brucei and Trypanosoma cruzi, Toxoplasma gondii, Eimeria spp., Neospora caninum, Sarcocystis spp., Plasmodium spp. (Plasmodium falciparum, Plasmodium vivax, Plasmodium ovale, Plasmodium malariae, Plasmodium knowlesi, for instance), and Cryptosporidium spp. It may also be chosen among Acanthamoeba spp., Babesia spp., Balantidium coli, Blastocysts, Dientamoeba fragiiis, Entamoeba histolytica, Giardia lamblia, Isospora belli, Leishmania spp., Naegleria fowleri, Rhinosporidium seeberi, Trichomonas vaginalis, Trypanosoma brucei, and Trypanosoma cruzi.
(iii) a bacterium selected from the strains Aeromonas hydrophila, Afipia felis, Actinomyces israelii, Actinobacillus actinomycetemcomitans, Achromobacter xylosoxidans, Acinetobacter baumannii, Bacillus anthracis, Bacillus cereus, Bartonella henselae, Bartonella ciarridgeiae, Bordetella pertussis (bacillus of Bordet and Gengou), Bordetella parapertussis, Bordetella bronchiseptica, Borrelia burgdorferi, Borrelia recurrentis, Brucella, Burkholderia cepacian, Burkholderia mallei, Burkholderia pseudomallei (Whitmore's bacillus), Campylobacter coli, Campylobacter fetus, Campylobacter jejuni, Cardiobacterium hominis, Chlamydia trachomatis, Chlamydophila pneumoniae, Chlamydophila psittaci, Clostridium botulinum, Clostridium difficile, Clostridium perfringens, Clostridium tetani, Corynebacterium diphtherias, Coxiella burnetii, Ehrlichia chaffeensis, Ehrlichia equi, Eikenella corrodens, Enterococcus: Enterococcus faecalis, Enterococcus faecium, Enterococcus gallinarum, Enterococcus flavescens, Enterococcus casseliflavus, Erysipelothrix rhusiopathiae, Escherichia coli, Enterobacteriaceae, Francisella tularensis, Haemophilus ducreyi, Haemophilus influenzae, Helicobacter pylori, Kingella kingae, Klebsiella granulomatis, Klebsiella oxytoca, Klebsiella ozenae, Klebsiella planticola, Klebsiella pneumoniae, Klebsiella rhinoscleromatis, Legionella pneumophila, Legionella longbeachae, Legionella micdadei, Leptospira interrogans, Listeria monocytogenes, Mycobacterium leprae (bacille de Hansen), Mycobacterium tuberculosis (Koch's bacillus), Mycobacterium bovis, Atypical mycobacteria (M. avium, M. bovis, M. intracellulare, etc.), Mycoplasma pneumoniae, Mycoplasma hominis, Mycoplasma genitalium, Neisseria gonorrhoeae (gonococcus), Neisseria meningitidis (meningococcus), Nocardia, Pantoea agglomerons, Pasteurella multocida, Plesiomonas shigelloides, Pneumococcus (common name of Streptococcus pneumoniae), Proteus mirabilis, Proteus vulgaris, Providencia stuartii, Pseudomonas aeruginosa, bacille pyocyanigue, Pseudomonads, Porphyromonas gingivalis, Rickettsia, Salmonella enterica (or salmonella), Serratia marcescens, Serratia proteamaculans, Shigella dysenteriae (or shigella), Shigella boydii, Shigella dysenteriae, Shigella flexneri, Shigella sonnei, Spirillum minus, Staphylocogues (Staphylococcus aureus, Staphylococcus epidermidis, Staphylococcus saprophyticus), Streptococcus pyogenes or Streptococci of group A, Streptococcus pneumoniae (or pneumococcus), Treponema pallidum, Ureaplasma urealyticum, Vibrio cholerae (cholerae and other varieties), Yersinia pestis, Yersinia enterocolitica, and Yersinia pseudotuberculosis.
(v) a fungus selected from Aspergillus fumigatus, Aspergillus flavus, Aspergillus clavatus, Blastomyces dermatitidis, Candida albicans, Candida auris, Coccidioides immitis, Cryptococcus neoformans, Cryptococcus gattii, Histoplasma capsulatum, Mucormycosis, Paracoccidioides brasihensis, Pneumocystis jirovecii, Pneumocystis Pneumonia, Sporothrix schenckii, Stachybotrys chartarum, and Talaromycosis.

The NPs used for the preparation of vaccine compositions form porous structures which are able to absorb the free antigens or to cover them so as to allow for their solubilization in an aqueous solution. Moreover, the proteins thus solubilized are stabilized.

The method according to the present disclosure thus makes it possible to solubilize lyophilized antigens in an aqueous solution, without using a solubilization aid.

The solution in which the lyophilized antigen is solubilized is, for example, an aqueous solution or a buffer solution, suitable for being used in the context of a vaccine use.

When the antigen is solubilized in a vaccine composition, it is understood that the composition may contain vaccine additives, indeed solubilization aids, without this calling into question the implementation of the method according to the present disclosure, when an antigen is added to the vaccine composition.

Within the meaning of the present disclosure, “solubilization aid,” also referred to as “lyophilization aid,” means an agent which allows for the solubilization of proteins as well as the stabilization of proteins. Examples are surfactants such as TWEEN®, EMIPIGEN®, triton, saccharides such as saccharose, polyols such as mannitol, inositol, polymers such as polyvinylpyrrolidone (PVP), etc.

Within the meaning of the present disclosure, “vaccine additive” means an immunomodulating agent. Such additives may be hydrophilic, such as oligodeoxynucleotides (CpG), or lipophilic, such as squalene, MPL, QS-21, etc.

For the avoidance of any confusion, it is explicitly intended that, within the context of this present disclosure, the NPs are not considered to be additives, but solubilizing agents and antigen release agents.

In order to re-suspend the lyophilized antigen, the protein/nanoparticle ration (weight/weight) is between 1/10 and 1/1. In a preferred embodiment, the protein/nanoparticle ratio is between 1/1 and 1/4, in particular, 1/3.

Following addition of the lyophilized antigens, the aqueous solution is incubated at ambient temperature. The incubation time may typically be between 3 minutes and 1 hour, preferably between 5 minutes and 30 minutes; it may be extended for 1 year without this affecting the method. Following incubation, the composition obtained may be either used directly as a vaccine composition, or used for solubilizing other lyophilized proteins.

Thus, in a particular embodiment of the present disclosure, at least two different lyophilized protein extracts are solubilized in the aqueous solution containing the nanoparticles. These different protein extracts may originate from different pathogens or contain antigens originating from different strains of the same pathogen.

The compositions obtained according to the present disclosure may be used as a vaccine in the veterinary field or in human health.

The present disclosure will be better understood upon reading the following examples, which are given by way of example and should under no circumstances be considered as limiting the scope of the present disclosure.

### EXAMPLES

### Example 1: Preparation of an Aqueous Composition of Hydrophilic Proteins from Lyophilized Proteins

A. Preparation of Lipidized Nanoparticles (NPL)

The NPLs are prepared as described previously in the patent EP2708237A1—2014 Mar. 19. 100 g maltodextrin is dissolved at ambient temperature in 2N sodium hydroxide, under magnetic stirring. The mixture is cross-linked by addition of epichlorohydrin, then cationized for one night by addition of GTMA in order to obtain a hydrogel. The hydrogel obtained is then neutralized with acetic acid, and crushed using a high-pressure homogenizer. The size of the particles thus obtained is determined by analysis of the dynamic light scattering (DLS). The particles are then purified by tangential flow ultrafiltration on a membrane of 750 kDa. The absence of salts and fragments of maltodextrin is controlled, respectively, by metering of silver nitrate and by DLS.

The resulting porous cationic nanoparticles of maltodextrin (referred to as NP+) are then charged with anionic phospholipids by injection of a solution of dipalmitoylphosphatidylglycerol (DPPG) prepared in solutol in order to obtain the nanoparticles referred to as “NPL.”

Non-cross-linked cationic nanoparticles of maltodextrin are prepared in the same way as the NP+ and NPLs, respectively; only the step of cross-linking by addition of epichlorohydrin is bypassed. The resulting non-cross-linked nanoparticles are referred to as “NPL NR” and “NP+ NR,” according to the fact that they are, respectively, charged with phospholipids or not.

B. Preparation of Antigens
The case of a purified antigen

The lyophilized bovine serum albumin (BSA), purified to 98%, originates from Sigma Aldrich (ref. A9647) and is used as a purified antigen model.
The case of a mixture of antigens from the total extract (ET) of E. coli

The strain E. coli NC 9001, originating from the bank “Public Health England,” is grown in an Erlenmeyer flask, while stirring, for 24 h at 37° C. in an LB medium. The bacteria are centrifugated for 15 minutes at 11000×g, then the centrifugate is washed by 4 successive rinses in sterile water. The bacterial centrifugate is then returned to 70% isopropanol and incubated in ice for 45 minutes in order to inactivate the bacteria. Following centrifugation for 15 minutes at 11000×g, the centrifugate is washed in sterile water and then placed in an ultrasonic bath for 10 minutes. Following centrifugation for 15 minutes at 11000×g, the centrifugate is dried under PSM for 30 minutes before freezing and then lyophilization. 10 mg lyophilisate are re-suspended in order to determine the dry weight/protein weight ratio by the μBCA method.

C. Preparation of an Extemporaneous Composition

The association of the antigens with the nanoparticles (NP+, NPL, NP+ NR and NPL NR) at ambient temperature (20-25° C.) is tested at different maturation times: 5 min, 30 min, 1 h and 24 h.

For each condition tested, 1 mg protein of antigen (BSA or ET E. coli) is placed in 2 ml NPL at 1.5 mg/ml, before being vortexed for 30 seconds in order to obtain a formulation having a ratio 1/3 (antigens/NPL). After 30 minutes of incubation, a sample metered at 1 mg protein (BSA or ET E. coli) is added to an aqueous solution in order to prepare the vaccine composition.

D. Methods of Analysis of the Association of Antigens with the NPLs

The association of the antigens to the NPLs is analyzed by:
DLS (dynamic light scattering, or diffraction of the polarized light), and zeta potential: measurement of the size (nm) and of the surface charge (mV) (free anionic antigens vs. cationic NPLs)
electrophoresis in non-denaturing conditions (native PAGE): the NPLs have too great a molecular mass to diffuse into the gel. Thus, in contrast with the free antigenic proteins, the proteins associated in the NPLs do not diffuse into the gel. The protein profile of the antigens formulated in the NPLs is controlled by electrophoresis in denaturing conditions (SDS PAGE).

### Example 2: Analysis of the Association of an Antigen (BSA) and/or a Total Extract of E. Coli with Different Nanoparticles

a. Association with NPLs

The association of the BSA with the NPLs is complete after 5 minutes of maturation at ambient temperature. The zeta potential of the BSA/NPL formulations is positive, and the size is greater than 20 nm (Table 1).

TABLE 1
DLS and zeta potential of BSA/NPL formulations

| | | | Z-av (nm) | Number (nm) | PDI | Zeta H2O (mV) | Comments |
|---|---|---|---|---|---|---|---|
| Before formulation | | | | | | | |
| NPL | | | 75 | 41 | 0.25 | +38 | Transparent |
| After formulation | | | | | | | |
| BSA 1/0 | 24 | h | 50 | 4 | 0.47 | −4 | Transparent |
| BSA/NPL 1/3 | | | 140 | 36 | 0.34 | +33 | |
| E. Coli 1/0 | | | 1400 | 1300 | 0.12 | −30 | Whitish + |
| E. Coli/NPL 1/3 | | | 160 | 37 | 0.39 | +31 | sedimentation |
| F1/3 | 5 | min | 245 | 35 | 0.50 | +32 | |
| BSA/NPL + E. Coli | 15 | min | 189 | 32 | 0.40 | +31 | |
| | 30 | min | 156 | 45 | 0.47 | +34 | |
| | 1 | h | 165 | 26 | 0.46 | +32 | |
| | 5 | h | 256 | 40 | 0.35 | +25 | |
| F1/3 | 5 | min | 150 | 31 | 0.47 | +32 | Whitish + |
| E. Coli/NPL + BSA | 15 | min | 130 | 33 | 0.44 | +20 | sedimentation |
| | 30 | min | 130 | 34 | 0.41 | +28 | |
| | 1 | h | 110 | 27 | 0.33 | +26 | |
| | 5 | h | 135 | 30 | 0.52 | +28 | |

The Z-av parameter represents the average size of the NPLs. Thus, it is observed that the BSA associating with the NPLs is very effective in less than 5 minutes.

The parameter “number” corresponds to the largest number of particles of a given size.

The PDI corresponds to the polydispersity index; it is recognized that the particles are monodispersed when this index is less than 0.3.

The zeta potential represents the surface charge of the nanoparticles. This is positive when the antigens (negatively charged) are absorbed inside the nanoparticle, or when the large antigens are covered by the nanoparticles.

Moreover, it is noted that adding a second protein to a formulation may be achieved in 5 minutes. The association of the BSA with the NPLs then of the total protein extract of E. coli is total after 5 minutes. The zeta potential of the formulations is positive, and confirms the association of the BSA and the ET E. coli with the NPLs (Table 1).

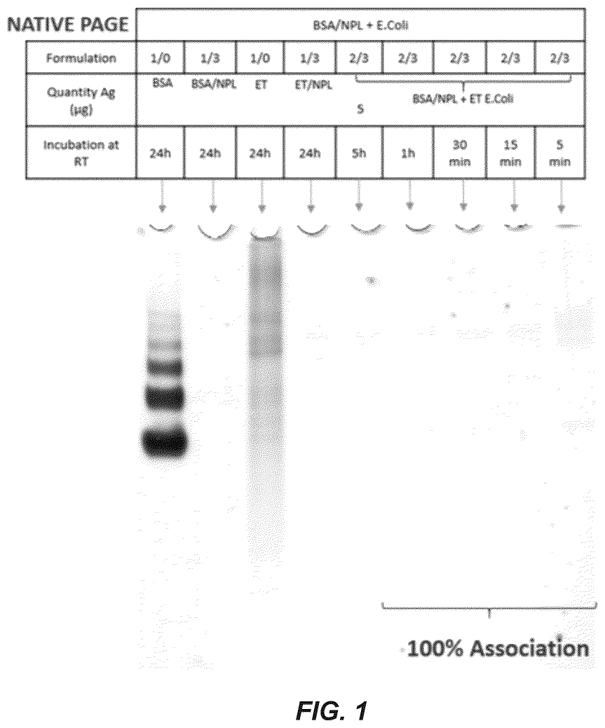

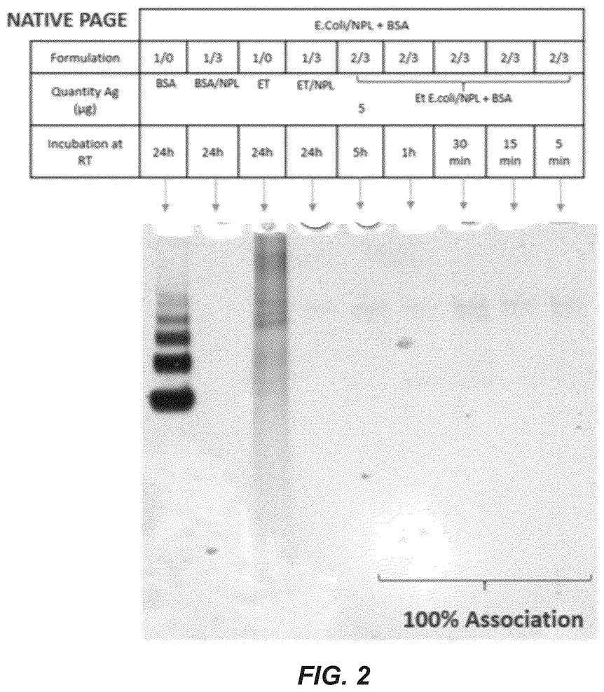

The analysis by electrophorese in native conditions confirms the total association of the BSA with the NPLs after 24 hours of maturation. Moreover, this analysis shows an association of the total extract of E. coli of 95%, from 5 minutes of maturation with the BSA/NPL (FIG. 1); a rapid and strong association is also observed when the BSA is associated with a total extract of E. coli/NPL (FIG. 2).

b. Association with NPL NRs

The association results are set out in Table 2.

TABLE 2
DLS & zeta potential of the non-cross-linked NPL formulations (NPL NR)

| | | | Z-av (nm) | Number (nm) | PDI | Zeta H2O (mV) | Comments |
|---|---|---|---|---|---|---|---|
| Before formulation | | | | | | | |
| NPL NR B | | | 45 | 20 | 0.23 | +30 | Transparent |
| After formulation | | | | | | | |
| BSA 1/0 | 24 | h | 50 | 4 | 0.47 | −4 | Transparent |
| BSA/NPL NR B 1/3 | | | 64 | 38 | 0.18 | +25 | |
| E. Coli 1/0 | | | 1400 | 1300 | 0.12 | −30 | Whitish + |
| E. Coli/NPL NR B 1/3 | | | 125 | 35 | 0.75 | +38 | sedimentation |
| F2/3 | 5 | min | 110 | 20 | 0.66 | +31 | |
| BSA/NPL NR B + E. Coli | 15 | min | 110 | 20 | 0.65 | +35 | |
| | 30 | min | 130 | 46 | 0.74 | +29 | |
| | 1 | h | 110 | 40 | 0.67 | +29 | |
| | 24 | h | 140 | 45 | 0.85 | +28 | |
| F2/3 | 5 | min | 120 | 46 | 0.73 | +28 | Whitish + |
| E. Coli/NPL NR B + BSA | 15 | min | 120 | 47 | 0.76 | +24 | sedimentation |
| | 30 | min | 110 | 44 | 0.68 | +30 | |
| | 1 | h | 180 | 17 | 1.0 | +30 | |
| | 24 | h | 120 | 52 | 0.63 | +35 | |

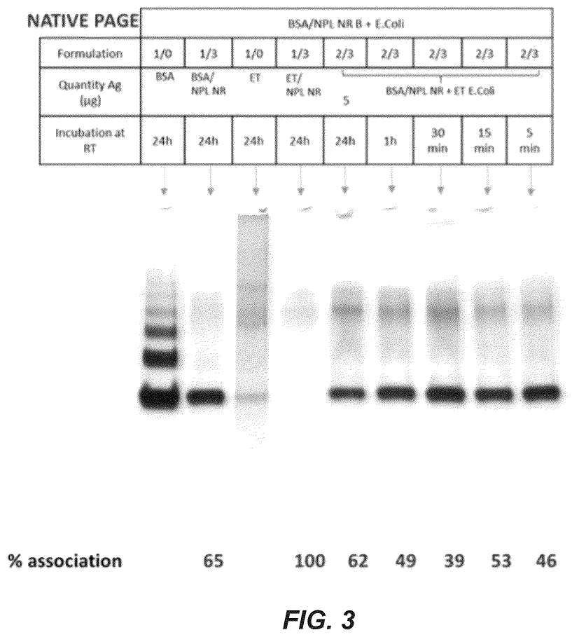

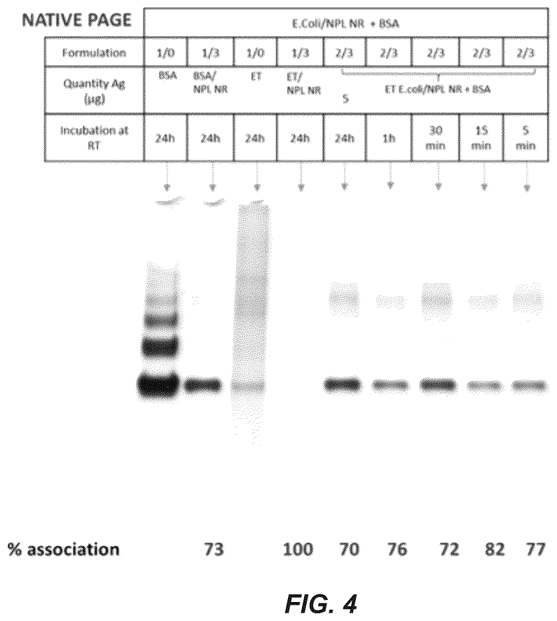

The association of the NPL NRs with the BSA or the ET of E. coli is observed following 24 h of incubation. Moreover, adding a second protein to an NPL NR formulation is possible even if the association of the BSA and the ET E. coli with the NPL NRs is less good than for the cross-linked NPLs. The zeta potential of the formulations is positive, and confirms the association of the BSA and the ET E. coli with the NPLs (Table 2). The analysis by electrophoresis in native conditions confirms these results (FIGS. 3 and 4).

c. Association with the NP+

The association results are set out in Table 3.

TABLE 3
DLS and zeta potential of NP+ formulations

| | | | Z-av (nm) | Number (nm) | PDI | Zeta H2O (mV) | Comments |
|---|---|---|---|---|---|---|---|
| Before formulation | | | | | | | |
| NP+ B | | | 91 | 39 | 0.24 | +38 | Transparent |
| After formulation | | | | | | | |
| BSA 1/0 | 24 | h | 50 | 4 | 0.47 | −4 | Transparent |
| BSA/NP+ 1/3 | | | 120 | 77 | 0.25 | +45 | |
| E. Coli 1/0 | | | 1400 | 1300 | 0.12 | +30 | Whitish + |
| E. Coli/NP+ 1/3 | | | 1240 | 1070 | 0.72 | +28 | sedimentation |
| F2/3 | 5 | min | 400 | 45 | 0.66 | +38 | Whitish + |
| BSA/NP+ + E. Coli | 15 | min | 940 | 45 | 1 | +37 | sedimentation |
| | 30 | min | 1450 | 93 | 0.95 | +38 | |
| | 1 | h | 860 | 78 | 0.90 | +33 | |
| | 24 | h | 550 | 56 | 0.91 | +33 | |
| F2/3 | 5 | min | 520 | 80 | 0.79 | +32 | Whitish + |
| E. Coli/NP+ + BSA | 15 | min | 220 | 76 | 0.74 | +34 | sedimentation |
| | 30 | min | 630 | 72 | 0.59 | +32 | |
| | 1 | h | 650 | 62 | 0.62 | +37 | |
| | 24 | h | 300 | 20 | 0.53 | +32 | |

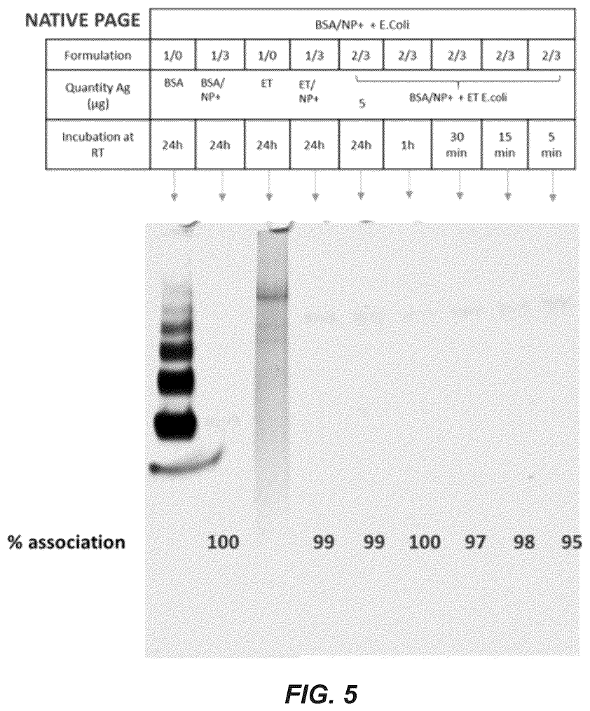

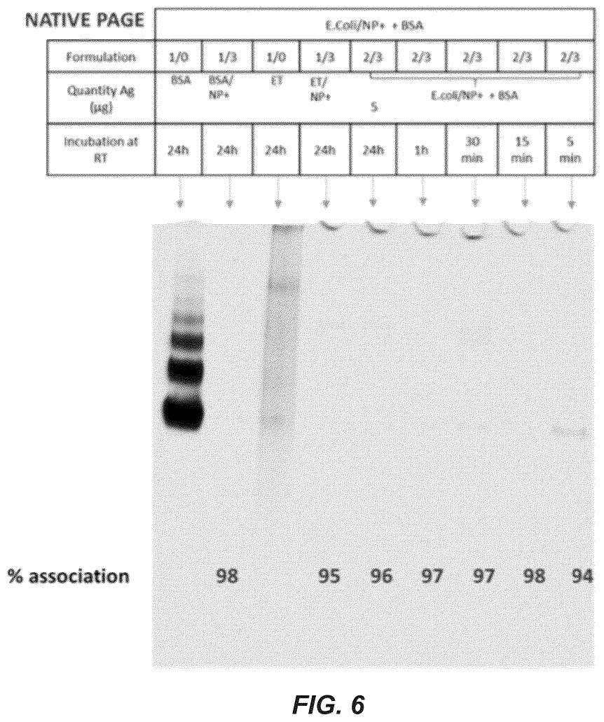

The association of the NPL NRs with the BSA or the ET of E. coli is observed following 24 h of incubation. Moreover, adding a second protein to an NP+ formulation can be achieved in 5 minutes, with an almost total association of the BSA and the ET E. coli with the NPL NRs. The zeta potential of the formulations is positive, and confirms the association of the BSA and the ET E. coli with the NP+ (Table 3). The analysis by electrophoresis in native conditions confirms these results (FIGS. 5 and 6).

d. Association with the NP+ NRs

The association results are set out in Table 4.

TABLE 4
DLS and zeta potential of NP+ NR formulations

| | | | Z-av (nm) | Number (nm) | PDI | Zeta H2O (mV) | Comments |
|---|---|---|---|---|---|---|---|
| Before formulation | | | | | | | |
| NP+ NR | | | 44 | 29 | 0.29 | +28 | Transparent |
| After formulation | | | | | | | |
| BSA 1/0 | 24 | h | 50 | 4 | 0.47 | −4 | Transparent |
| BSA/NP+ NR 1/3 | | | 62 | 12 | 0.38 | +22 | |
| E. Coli 1/0 | | | 1400 | 1300 | 0.12 | −30 | Whitish + |
| E. Coli/NP+ NR 1/3 | | | 1680 | 150 | 0.20 | +28 | sedimentation |
| F2/3 | 5 | min | 2240 | 825 | 0.44 | +30 | |
| BSA/NP+ NR + E. Coli | 15 | min | 2250 | 200 | 0.22 | +32 | |
| | 30 | min | 2200 | 1045 | 0.47 | +29 | |
| | 1 | h | 2180 | 1740 | 0.28 | +24 | |
| | 24 | h | 1440 | 1180 | 0.45 | +28 | |
| F2/3 | 5 | min | 1300 | 1325 | 0.22 | +20 | Whitish + |
| E. Coli/NP+ NR + BSA | 15 | min | 1380 | 1390 | 0.19 | +24 | sedimentation |
| | 30 | min | 1410 | 1430 | 0.17 | +24 | |
| | 1 | h | 1440 | 1420 | 0.24 | +27 | |
| | 24 | h | 1500 | 1140 | 0.29 | +27 | |

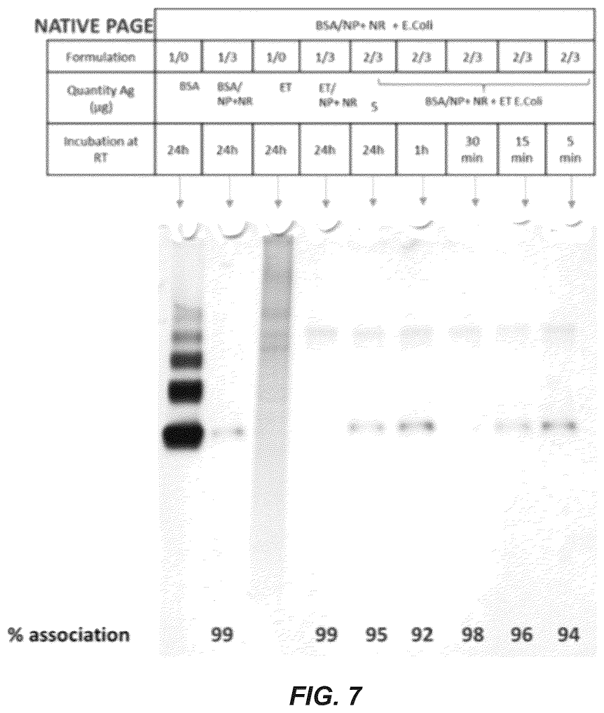

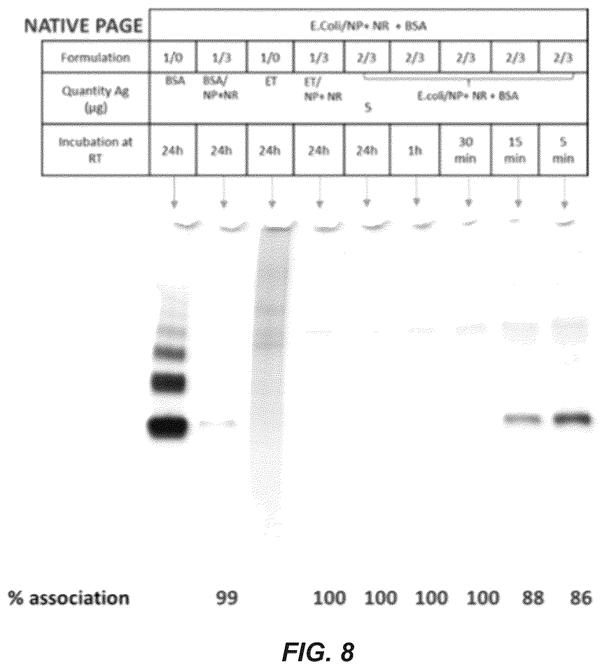

The association of the NP+ NRs with the BSA or the ET of E. coli is observed following 24 h of incubation. Moreover, adding a second protein to an NP+ NR formulation can be achieved in 5 minutes, with an almost total association of the BSA and the ET E. coli with the NP+ NRs. The zeta potential of the formulations is positive, and confirms the association of the BSA and the ET E. coli with the NP+ (Table 3). The analysis by electrophoresis in native conditions confirms these results (FIGS. 7 and 8).

### Conclusion

The extemporaneous formulations of lyophilized antigens with the 4 types of nanoparticles studied are achieved in less than 30 minutes.

These results show:
on the one hand, the ability to formulate a lyophilized antigen in an aqueous solution (solubilization) in order to prepare a vaccine composition that is ready for use
on the other hand, that it is possible to add one or more antigens (one or more valencies) to a vaccine composition which already comprises one or more formulated antigens; this or these additional antigens are solubilized in a pre-existing vaccine composition, to which nanoparticles have previously been added.

1. A method for preparing a vaccine composition from at least one lyophilized antigen, comprising the steps of:
providing an aqueous solution comprising a cationic nanoparticle consisting of a porous cationic polysaccharide core;
adding the lyophilized antigen to the aqueous solution; and
incubating the composition thus obtained at ambient temperature.

2. The method of claim 1, wherein the aqueous solution comprises at least one antigenic protein.

3. The method of claim 1, wherein the porous cationic polysaccharide core is non-cross-linked and is obtained by a reaction between a polysaccharide selected from starch, dextran, dextrin and maltodextrin, poly-fructoses (inulin), poly-mannoses, poly-galactoses, poly-galactomannans (guar gum), and at least one cationic ligand selected from a primary, secondary or tertiary amine, and quaternary ammoniums.

4. The method of claim 1, wherein the porous cationic polysaccharide core is cross-linked and is obtained by a reaction between a polysaccharide selected from starch, dextran, dextrin and maltodextrin, poly-fructoses (inulin), poly-mannoses, poly-galactoses, poly-galactomannans (guar gum), and at least one cationic ligand selected from a primary, secondary or tertiary amine, and quaternary ammoniums, and addition of a cross-linking agent.

5. The method of claim 4, wherein the cross-linking agent is selected from epichlorohydrin, a dicarboxylic acid or an acid chloride.

6. The method of claim 3, wherein the cationic polysaccharide core is obtained by reaction between a maltodextrin and a glycidyltrimethylammonium.

7. The method of claim 1, wherein the porous cationic polysaccharide core is charged with an anionic phospholipid.

8. The method of claim 7, wherein the anionic phospholipid is diacylphosphatidyl glycerol, diacylphosphatidyl serine, or diacylphosphatidyl inositol.

9. The method of claim 8, wherein the anionic phospholipid is dipalmitoylphosphatidylglycerol.

10. The method of claim 1, wherein the porous cationic polysaccharide core is not charged with lipids.

11. The method of claim 1, wherein the lyophilized antigen is an antigenic protein or a mixture of antigenic proteins.

12. The method of claim 11, wherein the mixture of antigenic proteins is made up of one or more purified antigens or a pathogen extract.

13. The method of claim 12, wherein the pathogen is selected from a parasite, a virus, a bacterium, a mycobacterium, and a fungus.

14. The method of claim 13, wherein the pathogen is:
(i) a virus selected from herpes simplex virus 1 and 2, human papillomavirus, cytomegalovirus, mycobacterium tuberculosis, dengue fever, HIV, respiratory syncytial virus (RSV), hepatitis A virus, hepatitis B virus, hepatitis C virus, SARS-Cov2, rabies virus, African horse sickness virus, African swine fever virus, Andes virus, avian influenza virus, equine influenza virus, bluetongue virus, Chapare virus, Chikungunya virus, Choclo virus, Crimean-Congo hemorrhagic fever virus, dengue virus, Dobrava-Belgrade virus, eastern equine encephalitis virus, Ebola virus, foot-and-mouth disease virus, capripox virus, Guanarito virus, Hantaan virus, Hendra virus (equine morbillivirus), porcine herpesvirus (Aujeszky's disease virus), equine herpesvirus, classical swine fever virus, Japanese encephalitis virus, Junin virus, Kyasanur forest disease, Laguna Negra virus, Lassa fever virus, louping ill virus, Lujo virus, lumpy skin disease virus, lymphocytic choriomeningitis virus, Machupo virus, Marburg virus, monkeypox virus, Murray Valley encephalitis virus, Newcastle disease virus, Nipah virus, Omsk hemorrhagic fever virus, Oropouche virus, ovine rinderpest virus, porcine enterovirus 9 (swine vesicular disease virus), Powassan encephalitis virus, Rift Valley fever virus, rinderpest virus, Rocio virus, Sabia virus, Seoul virus, sheep pox virus, Sin Nombre virus, Saint Louis encephalitis virus, Teschen disease virus (enterovirus encephalomyelitis), porcine reproductive and respiratory syndrome virus, tick-borne encephalitis virus (Russian Spring-Summer encephalitis virus), smallpox virus, Venezuelan equine encephalitis virus, vesicular stomatitis virus, Western equine encephalitis virus, yellow fever virus, and feline leukemia virus, or
(ii) an intracellular parasite selected from Acanthamoeba spp., Babesia spp., Balantidium coli, Blastocytis, Dientamoebafragiiis, Entamoebahistolytica, Giardia lemblia, Isospora belli, Leishmania spp., Naegleriafowleri, Rhinosporidiumseeberi, Trichomonas vaginalis, Trypanosoma brucei, Trypanosoma cruzi, Toxoplasma gondii, Eimeria spp., Neospora caninum, Sarcocystis spp., Plasmodium spp. and Cryptosporidium spp., or
(iii) a bacterium selected from the strains Aeromonas hydrophila, Afipia felis, Actinomyces israelii, Actinobacillus actinomycetemcomitans, Achromobacter xylosoxidans, Acinetobacter baumannii, Bacillus anthracis, Bacillus cereus, Bartonella henselae, Bartonella ciarridgeiae, Bordetella pertussis (bacillus of Bordet and Gengou), Bordetella parapertussis, Bordetella bronchiseptica, Borrelia burgdorferi, Borrelia recurrentis, Brucella, Burkholderia cepacian, Burkholderia mallei, Burkholderia pseudomallei (Whitmore's bacillus), Campylobacter coli, Campylobacter fetus, Campylobacter jejuni, Cardiobacterium hominis, Chlamydia trachomatis, Chlamydophila pneumoniae, Chlamydophila psittaci, Clostridium botulinum, Clostridium difficile, Clostridium perfringens, Clostridium tetani, Corynebacterium diphtherias, Coxiella burnetii, Ehrlichia chaffeensis, Ehrlichia equi, Eikenella corrodens, Entcrococcus: Enterococcus faecalis, Enterococcus faecium, Enterococcus gallinarum, Enterococcus flavescens, Enterococcus casseliflavus, Erysipelothrix rhusiopathiae, Escherichia coli, Enterobacteriaceae, Francisella tularensis, Haemophilus ducreyi, Haemophilus influenzae, Helicobacter pylori, Kingella kingae, Klebsiella granulomatis, Klebsiella oxytoca, Klebsiella ozenae, Klebsiella planticola, Klebsiella pneumoniae, Klebsiella rhinoscleromatis, Legionella pneumophila, Legionella longbeachae, Legionella micdadei, Leptospira interrogans, Listeria monocytogenes, Mycobacterium leprae (bacille de Hansen), Mycobacterium tuberculosis (Koch's bacillus), Mycobacterium bovis, (Mycobacterium avium, Mycobacterium bovis, Mycobacterium intracellulare, Mycoplasma pneumoniae, Mycoplasma hominis, Mycoplasma genitalium, Neisseria gonorrhoeae (gonococcus), Neisseria meningitidis (meningococcus), Nocardia, Pantoea agglomerons, Pasteurella multocida, Plesiomonas shigelloides, Streptococcus pneumoniae, Proteus mirabilis, Proteus vulgaris, Providencia stuartii, Pseudomonas aeruginosa, bacille pyocyanigue, Pseudomonades, Porphyromonas gingivalis, Rickettsia, Salmonella enterica, Serratia marcescens, Serratia proteamaculans, Shigella dysenteriae, Shigella boydii, Shigella dysenteriae, Shigella flexneri, Shigella sonnei, Spirillum minus, Staphylococcus aureus, Staphylococcus epidermidis, Staphylococcus saprophyticus, Streptococcus pyogenes, Streptococci of group A, Treponema pallidum, Ureaplasma urealyticum, Vibrio cholerae, Yersinia pestis, Yersinia enterocolitica, and Yersinia pseudotuberculosis, or
(iv) a fungus selected from Aspergillus fumigatus, Aspergillus flavus, Aspergillus clavatus, Blastomyces dermatitidis, Candida albicans, Candida auris, Coccidioides immitis, Cryptococcus neoformans, Cryptococcus gattii, Histoplasma capsulatum, Mucor, Paracoccidioides brasiliensis, Pneumocystis jirovecii, Sporothrix schenckii, Stachybotrys chartarum, and Talaromyces.
